(19) United States
(12) Patent Application Publication
(10) Pub. No.: US 2021/0338876 A1
(43) Pub. Date: Nov. 4, 2021

(54) Charging Device for Charging an Electrical Energy Store of a Motor Vehicle

(71) Applicant: Daimler AG, Stuttgart (DE)

(72) Inventors: Matthias KRONEWITTER, Esslingen (DE); Christoph SCHWILL, Stuttgart (DE)

(73) Assignee: Daimler AG, Stuttgart (DE)

(21) Appl. No.: 17/271,890

(22) PCT Filed: Aug. 1, 2019
(86) PCT No.: PCT/EP2019/070763

§ 371 Date: Feb. 26, 2021

(30) Foreign Application Priority Data
Aug. 27, 2018 (DE) 10 2018 006 774.3

(51) Int. Cl. A61L 9/12; B60L 53/302; B60L 53/16; B60L 53/60

(52) U.S. Cl. CPC A61L 9/122; B60L 53/302; A61L 2209/134; B60L 53/60; A61L 2209/133; B60L 53/16

(57) ABSTRACT

A charging device for charging an electrical energy store of a motor vehicle includes a fragrance dispensing device, where a fragrance is dispensable into a surrounding of the charging device by the fragrance dispensing device. A method for charging an electrical energy store of a motor vehicle includes dispensing a fragrance into a surrounding of a charging device by a fragrance dispensing device integrated in the charging device.

### BACKGROUND AND SUMMARY OF THE INVENTION

The invention relates to a charging device for charging an electrical energy store of a motor vehicle.

The invention further relates to a method for charging an electrical energy store of a motor vehicle.

A charging system is known from DE 10 2012 205 972 A1, comprising a vehicle having a charging socket and a circuit breaker unit and comprising a charging device for charging an electrical energy store of the vehicle on an AC mains power supply external to the vehicle. The charging device comprises a housing which has outer cooling ribs and is penetrated by a cooling channel, which is set up for a current of air through the housing.

The object of the invention is to specify a charging device, improved in comparison to the prior art, for charging an electrical energy store of a motor vehicle and a method for charging an electrical energy store of a motor vehicle.

According to the invention, the charging device for charging an electrical energy store of a motor vehicle comprises a fragrance dispensing device for dispensing at least one fragrance into the surroundings of a charging device. By means of fragrancing of the surroundings of the charging device, the charging device makes possible a fragrancing of a space, for example a garage, without an additional and separate fragrance dispensing device being necessary.

According to a possible design of the charging device, the fragrance dispensing device comprises at least one distribution unit for distributing the fragrance, wherein the distribution unit is arranged in such a way that the distributed fragrance can be distributed into an airflow generated by means of at least one fan of a cooling device. This enables an active and effective conveyance of the fragrance into the surroundings of the charging device while simultaneously cooling an energy converter of the charging device, for example a power electronic system of the energy converter.

According to a further possible design of the charging device, the fragrance dispensing device comprises at least one storage container for stockpiling the fragrance, wherein the at least one storage container is formed as an exchangeable cartridge. Thus, a simple exchange of the storage container is possible for a user.

According to a possible development of the charging device, it is provided that it comprises a housing, inside which and/or on which a source port for coupling with an electrical energy source, a charging port for coupling with the vehicle, an energy converter for converting energy of the energy source into charging energy for the charging port and the fragrance dispensing device are arranged. Here, the housing is set up to be mounted on a building wall, a building ceiling or a building floor. On the one hand, the housing makes it possible to protect components of the charging device and, on the other hand, makes a variety of different mounting possibilities possible.

In a possible design of the charging device, it is provided that the charging port comprises a primary coil which is set up for inductive energy transfer to a secondary coil on the side of the vehicle. Thus, an inductive and subsequently contactless and comfortable charging of the energy store is possible.

In a further possible design of the charging device, an operating arrangement is provided which is set up for programming and/or controlling a charging process of the electrical energy store and/or the fragrance dispensing device. This makes it possible for a user to be able to also be able to simply undertake programming and controlling of the air fragrancing device, along with programming and controlling of the charging process. Here, a period of time for the fragrancing, for example independent of a period of time for the charging process and independent of a presence of a motor vehicle, at least one fragrance and/or a series of fragrances, for example, can be predetermined.

In a possible development of the charging device, a mobile terminal is provided with a piece of application software which is set up for programming and/or controlling a charging process of the electrical energy store and/or the fragrance dispensing device. This makes a remote control and/or remote programming of the charging process of the electrical energy store and/or the fragrance dispensing device possible.

In the method according to the invention for charging an electrical energy store of a motor vehicle, at least one fragrance is dispensed into the surroundings of the charging device by means of a fragrance dispensing device integrated in a charging device. The method makes it possible to fragrance a space, for example a garage, by fragrancing the surroundings of the charging device.

In a possible design of the method, ventilation of the motor vehicle is activated and air containing the fragrance is led out of the surroundings of the charging device into an interior space of a vehicle. Thus it is possible to also supply the interior space of the vehicle with the air containing the fragrance in addition to the fragrancing of the space.

Exemplary embodiments of the invention are explained in more detail below by means of drawings.

### BRIEF DESCRIPTION OF THE DRAWINGS

FIG. 1 shows, schematically, a perspective view of a cut-out of a charging device; and

FIG. 2 shows, schematically, a side view of a motor vehicle and a charging device.

### DETAILED DESCRIPTION OF THE DRAWINGS

Parts corresponding to one another are provided with the same reference numeral in all figures.

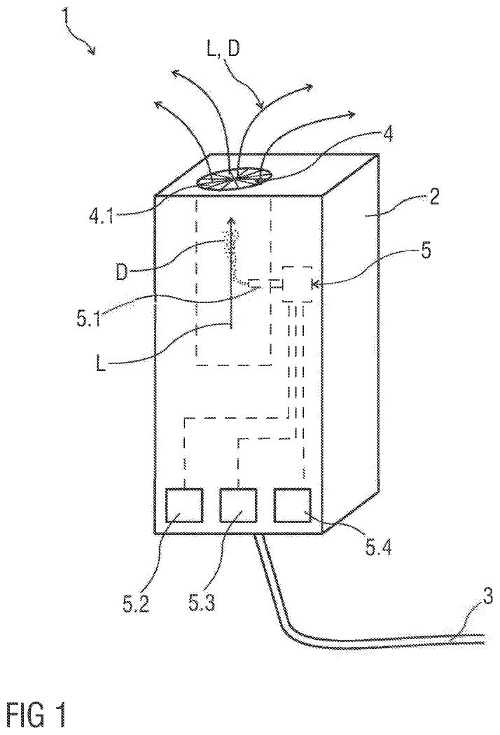

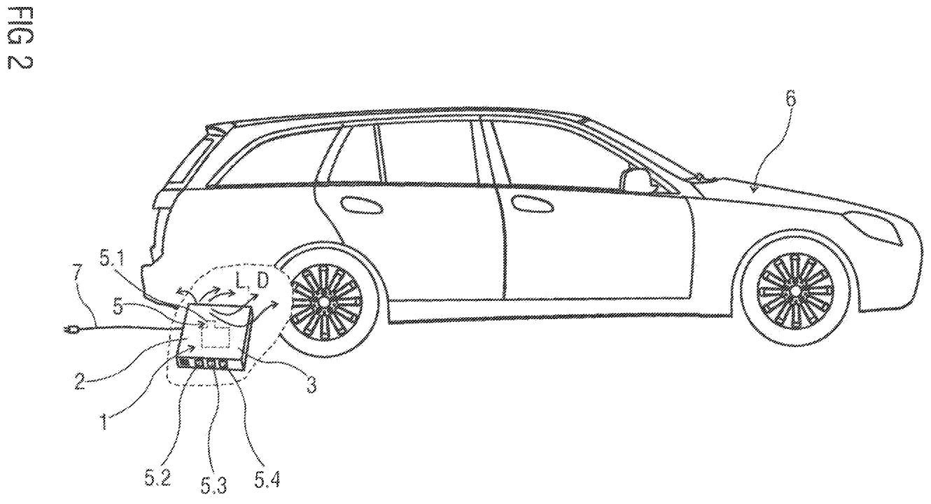

A perspective view of a cut-out of a possible exemplary embodiment of a charging device 1 for an electrical energy store of a motor vehicle 6 shown in FIG. 2 is depicted in FIG. 1. The motor vehicle 6 is, for example, a motor vehicle 6 having an electric drive or a hybrid drive.

Here, the charging device 1 is formed as a wall charging station and comprises a housing 2, which is set up for mounting on a building wall. A source port 7 not depicted in more detail in FIG. 2 for coupling with an electrical energy source, for example a public AC mains power supply, a charging port 3 for coupling with the vehicle 6 and an energy converter for converting energy of the energy source into charging energy for the charging port 3 are arranged inside the housing 2 or on it.

Here, the charging port 3 comprises, for example, a plug or a bushing for coupling with the motor vehicle 6 or is a primary coil, which is set up for inductive energy transfer to a secondary coil on the side of the vehicle and is arranged, for example, on or inside a building floor. For this purpose, the primary coil is coupled with the energy converter arranged in the housing 2 by means of a cable. The motor vehicle 6 is positioned with its secondary coil above the primary coil to carry out the charging process.

Furthermore, the charging device 1 comprises a cooling device 4 for cooling the energy converter, in particular a power electronic system thereof, which generates thermal losses during the energy conversion. The cooling device 4 comprises a fan 4.1, which generates an airflow L for cooling, in order to divert heat from the housing 2.

In addition, the charging device 1 comprises a fragrance dispensing device 5, which is arranged inside the housing 2. The fragrance dispensing device 5 comprises a distribution unit 5.1, for example an atomizer, for distributing a fragrance D into the airflow L generated by means of the fan 4.1.

The distribution unit 5.1 is coupled with several storage containers 5.2 to 5.4, respectively formed as exchangeable cartridges, for stockpiling the fragrance D, wherein the storage containers 5.2 to 5.4 are received in the housing 2.

When the fragrance dispensing device 5 is activated, the fragrance D is thus dispensed into the surroundings of the charging device. Different fragrances D, in particular, are contained in the storage containers 5.2 to 5.4, wherein a simultaneous distribution of several fragrances D into the airflow L is possible in a possible design.

Here, the activation of the fragrance dispensing device 5 can selectively be carried out depending on the execution of the charging process or independently thereof.

Controlling and/or programming the charging process and the fragrance dispensing device 5 is carried out by means of an operating arrangement not depicted in more detail. The operating arrangement is formed, for example, as a touch-sensitive screen. Alternatively or additionally, the control and/or programming of the charging process and the fragrance dispensing device 5 is carried out by means of a mobile terminal, for example a smartphone, with a corresponding piece of application software. Here, a user can predetermine a period of time of the charging process, for example, and a period of time of the fragrancing as part of the control and/or programming, in particular independently of the period of time of the charging process and independently of a presence of the motor vehicle 6. Furthermore, the user can predetermine which fragrance D is to be distributed into the airflow L from which storage container 5.2 to 5.4, whether several fragrances D are to be distributed simultaneously or whether different fragrances D are to be distributed in a predetermined sequence. Thus, it is possible for the user to have a personalized room fragrance generated for a garage, for example, and to program and to control this.

In a possible design, a ventilation of the motor vehicle 6 is additionally activated during the distribution of the at least one fragrance D into the surroundings of the charging device and thus the air containing the fragrance D is led from the surroundings of the charging device into an interior space of the vehicle. This function can also be controlled and/or programmed by the user via the operating arrangement and/or the mobile terminal, wherein, to do so, there is a communication connection between the motor vehicle 6 and the charging device 1.

FIG. 2 shows a side view of the motor vehicle 6 and a possible further exemplary embodiment of a charging device 1.

In contrast to the exemplary embodiment of the charging device 1 depicted in FIG. 1, the charging device 1 is not formed as a wall charging device, but rather completely integrated in a building floor or arranged on this and formed for the inductive charging of the energy store.

The charging port 3 here comprises the primary coil which is set up for the inductive energy transfer to the secondary coil on the side of the vehicle. Furthermore, the source port 7 is formed as a cable with a plug for coupling with the energy source.

The distribution of the at least one fragrance D into the surroundings of the charging device is here carried out analogously to the description of FIG. 1, yet with the difference that the at least one fragrance D is emitted in the region of the building floor and, when the motor vehicle 6 is correspondingly positioned, below this. With corresponding controlled ventilation of the motor vehicle 6, an effective supply of the at least one fragrance D into the interior space of the vehicle is possible.

1.-9. (canceled)

10. A charging device for charging an electrical energy store of a motor vehicle, comprising:
a fragrance dispensing device, wherein a fragrance is dispensable into a surrounding of the charging device by the fragrance dispensing device.

11. The charging device according to claim 10, wherein the fragrance dispensing device includes a distribution unit, wherein the fragrance is distributable by the distribution unit, and wherein the distribution unit is disposed such that the fragrance is distributable into an airflow generated by a fan of a cooling device of the charging device.

12. The charging device according to claim 10, wherein the fragrance dispensing device includes a storage container for storing the fragrance and wherein the storage container is an exchangeable cartridge.

13. The charging device according to claim 10 further comprising a housing, wherein inside the housing and/or on the housing is disposed:
a source port for coupling with an electrical energy source;
a charging port for coupling with the motor vehicle:
an energy converter for converting energy of the electrical energy source into a charging energy for the charging port; and
the fragrance dispensing device;
wherein the housing is configured for mounting on a building wall, a building ceiling, and/or a building floor.

14. The charging device according to claim 13, wherein the charging port includes a primary coil which is configured for inductive energy transfer to a secondary coil on a side of the motor vehicle.

15. The charging device according to claim 10 further comprising an operating arrangement which is configured for programming and/or controlling a charging process of the electrical energy store and/or the fragrance dispensing device.

16. The charging device according to claim 10 further comprising a mobile terminal which is configured for programming and/or controlling a charging process of the electrical energy store and/or the fragrance dispensing device.

17. A method for charging an electrical energy store of a motor vehicle, comprising the step of:
dispensing a fragrance into a surrounding of a charging device by a fragrance dispensing device integrated in the charging device.

18. The method according to claim 17 further comprising the steps of activating a ventilation of the motor vehicle and leading air containing the fragrance out of the surrounding of the charging device into an interior space of the motor vehicle by the ventilation.
